(19) United States
(12) Patent Application Publication
(10) Pub. No.: US 2011/0301614 A1
(43) Pub. Date: Dec. 8, 2011

(54) PENTAGONAL SCREWDRIVER, FOR SCREWING SCREWS WITH PENTAGONAL RECESSES, IN PARTICULAR ORTHOPAEDIC SCREWS

(75) Inventor: Frédéric Impellizzeri, Salon de Provence (FR)

(73) Assignee: BIOTECH INTERNATIONAL (SOCIETE PAR ACTIONS SIMPLIFIEE), Salon De Provence (FR)

(21) Appl. No.: 13/146,153

(22) PCT Filed: Jan. 26, 2010
(86) PCT No.: PCT/FR2010/000064

§ 371 Date: Aug. 23, 2011

(30) Foreign Application Priority Data
Feb. 23, 2009 (FR) 09/00808

(51) Int. Cl. A61B 17/56

(52) U.S. Cl. 606/104

(57) ABSTRACT

A pentagonal screwdriver, for screwing screws with pentagonal recesses, in particular orthopaedic screws with one face thereof tilted relative to and in the direction of the longitudinal axis of said screwdriver, the other four faces of the screwdriver being straight and parallel to said longitudinal axis.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention concerns a pentagonal screwdriver for screwing screws with pentagon-shaped heads or screws with five socket flats, in particular for screws usable in orthopedic surgery, such as hollow spindle-guided screws. It also aims at a system of screw works usable in particular in orthopedic surgery, featuring such a screwdriver and screws with pentagonal recesses.

2. Description of Related Art Including Information Disclosed Under 37 CFR 1.97 and 37 CFR 1.98

Screws with polygonal recesses are well known and widely used as elements for fastening or assemblies, especially hex screws. Such screws are used in various industries and notably in orthopedic surgery to reunite bone fragments, in order to achieve the reduction of osseous fractures through osteosynthesis. The rotational drive of these screws is obtained by means of screwdrivers or keys presenting a working end with a section that is complementary to that of the recess of the screws.

The main disadvantage resulting from the use of hex screws and hex screwdrivers is that since the angles at the apex of a hexagon are 120°, the phenomenon of the screwdriver shifting in the recess occurs more easily. As a matter of fact, the more flats there are and the larger the angles, the more the geometrical shape tends towards the circle in which it is inscribed. There is hence little material to be deformed by the screwdriver to distort the recess of the screw head. The phenomenon of shifting is being amplified by the fact that in order to be able to insert the screwdriver into the recess of the screw, there necessarily has to be some play between the flats with respect to said screwdriver and said recess.

Furthermore, the stress is transferred through the six vertical edges of the screwdriver which wear down and deform the recess of the screw, the consequence of which may be that it will no longer be possible to remove the screw from the bone material into which it has been screwed.

The obvious solution to these disadvantages would be to create screws with a recess featuring a number of flats below 6. In fact, by reducing the value of the apex angles of the polygon (pentagon, square, triangle), it reduces or suppresses the shifting of the screwdriver shaped in complementary manner.

However, this solution is not applicable to the majority of screws used in orthopedic surgery, on account of the fact that the heads of these screws present, in their proximal portion, a very reduced diameter, generally in the order of 2 mm to 3 mm, in as much as these heads present, most of the time, a conical shape and that their diameter therefore diminishes progressively.

If one considers, for example, a system of screw works using screws with a square recess and a screwdriver shaped in complementary manner, the angles at the apex of a square being 90°, there is good transfer of stress. However, for a section equivalent to that of a hexagon, the square is inscribed in a circle of a larger diameter, and this all the more so, when its section is reduced by the value of the section of the guiding spindle. Thus, to be able to use a screw with a square recess, it requires a screw with a much bigger diameter than that of screws with a hexagonal recess. Now, in orthopedic surgery the heads of screws measure, as previously stated, from 2 to 3 mm. They can therefore have only very small square recesses where the distance between opposite flats would be close to the bore diameter receiving the guiding spindle, and in this case, taking into account the resulting very weak section, the screwdriver and/or the head of screw would not withstand the effect of the turning force.

Triangular recesses which would transfer the stress even better because of their 60° angles, would present the same disadvantage as the square recesses, in any application to orthopedic screws.

A good compromise, permitting to have the smallest angles possible, in order to transfer the stress torque, and the largest possible section of recess and end of the screwdriver, so that the latter will not break under the torsion effect and the recess of the screw head will not be deformed, would therefore be the utilization of screws with a pentagonal recess and of a pentagonal screwdriver.

Nuts with pentagonal recess and pentagonal drive wrenches have already been proposed.

For example, document JP-10.146.772 describes an Allen wrench with a cross section of pentagonal shape and a fastening screw with pentagonal recess which permit reducing the abrasion of the distal end of the wrench which is inserted into the recess of the nut.

Document GB-2.109.079 describes attaching nuts presenting a tail with a threaded axial bore and a conical head with a hexagonal or pentagonal hole, essentially used to ensure the fastening of U-bolts for metallic children's play structures providing secure fastening while avoiding sharp edges and which can be used with standard wrenches.

However, the use, by itself, of screw work systems featuring pentagonal wrenches or screwdrivers and screws with pentagonal recess, does not present a very significant progress relative to systems using screwdrivers and screws with hexagonal recess, as one does not know such systems being actually applied in industry and that they are not applied in the domain of orthopedic surgery.

Furthermore, in such screw work systems the generating line which transfers the stress is constituted by the five vertical edges. There still exists a shifting effect although it is less pronounced than for hexagonal screwdrivers because there is more material to deform.

One aim of the present invention is to remedy the aforementioned disadvantages by offering a reliable and precise solution to permit a more effective and more secure placement of screws, in particular in orthopedic surgery.

### BRIEF SUMMARY OF THE INVENTION

According to the invention, this aim has been achieved because of a pentagonal screwdriver with at least one flat being inclined in the direction of the axis of said screwdriver.

The screwdriver according to the invention provides several interesting advantages. It makes it possible in particular:
to compensate for play which exists between a screwdriver and the recess of a screw head;
to avoid shifting by firmly applying the flats of the screwdriver which are opposite the inclined flat against the corresponding faces of the recess of the screw head;
to transfer the stress through the intermediary of an edge, of two flats and one angle; and
to have a gripping effect which permits holding the screw of the screwdriver.

According to an advantageous embodiment, the inclined flat of the screwdriver presents an incline between 2° and 3°.

According to a preferred embodiment, the inclined flat presents an inclination of 2° for use with screws featuring a head diameter of 2 mm or less than 2 mm, and an inclination of 3° for use with screws featuring a head diameter above 2 mm.

According to another characteristic arrangement, the screwdriver according to the invention presents an axial bore permitting the passage of a piercing spindle used for performing certain operations in orthopedic surgery.

According to a preferred embodiment, the screwdriver is made of any so-called surgical material presenting the necessary hardness.

According to an advantageous embodiment, the screwdriver according to the invention is made of stainless steel having undergone a heat treatment with the aim of increasing its hardness.

### BRIEF DESCRIPTION OF THE DRAWINGS

The aforementioned aims, characteristics and advantages, and others will become clearer from the description which follows and the attached drawings in which:

FIG. 1 is a perspective view of an embodiment of the pentagonal screwdriver according to the invention.

FIG. 2 is a front view and at a larger scale of the distal part of the pentagonal screwdriver, illustrating the inclined flat.

FIG. 3 is a view of the underside and at a larger scale of the distal part of the screwdriver.

FIG. 4 is a detailed section view and at a larger scale, of a pentagonal screwdriver as it is being inserted into a screw head with a pentagonal recess.

FIG. 5 is an analog view to FIG. 4 and shows the screwdriver completely inserted into the screw head with pentagonal recess.

FIG. 6 is a view from above in a section along the line 6-6 of FIG. 5 showing the edges or the surfaces which transfer the screwing torque.

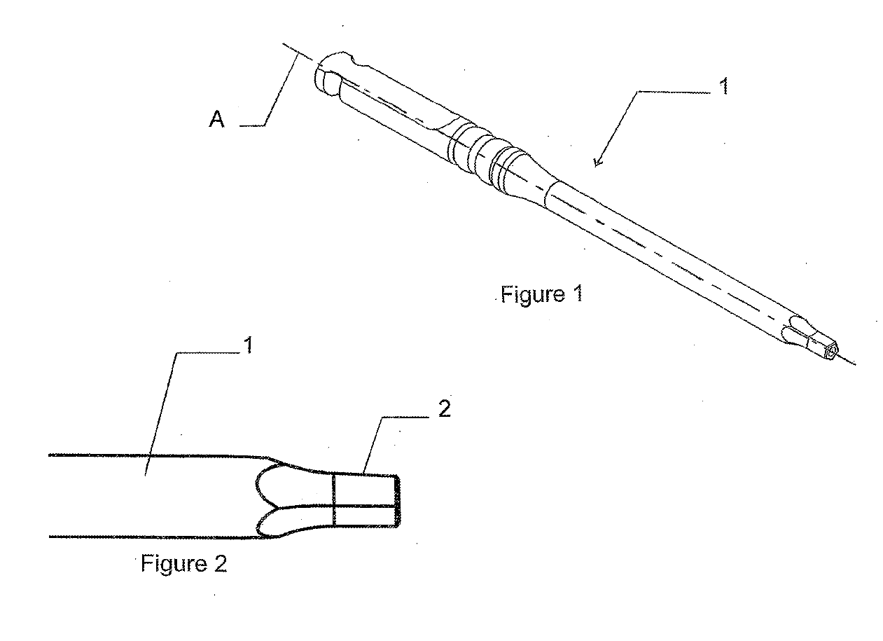

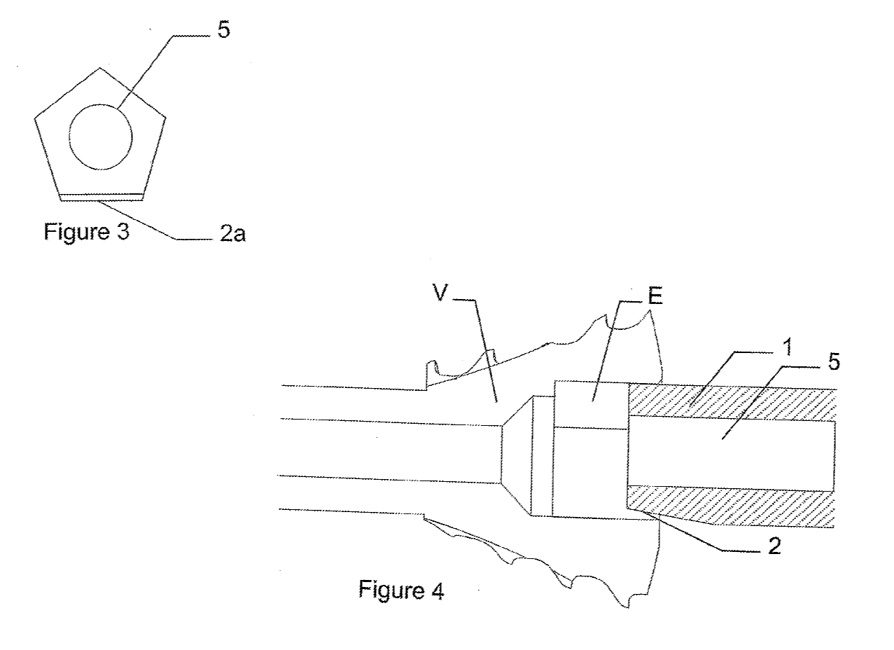

Reference is made to the drawings to describe an interesting, although by no means limiting example, of the pentagonal screwdriver according to the invention.

### DETAILED DESCRIPTION OF THE INVENTION

The following description and claims are limited to a screwdriver but the device according to the invention can also be adapted to a wrench or to any other appropriate fastening system.

The pentagonal screwdriver 1 according to the invention features a distal drive end with five flats of which at least one flat 2 is inclined in relation to the longitudinal axis A of the screwdriver and in the direction of that axis.

According to an advantageous embodiment, said inclined face 2 presents an inclination between 2° and 3°.

Preferably, said inclination amounts to 2° for use with screws featuring a diameter of 2 mm or less than 2 mm, and 3° for use with screws featuring a head with a diameter above 2 mm.

According the example shown the pentagonal screwdriver 1 presents an axial bore permitting the passage of a guiding spindle.

According to an advantageous embodiment the screwdriver 1 is made of any so-called surgical material presenting the necessary hardness, for example of stainless steel.

According to the example shown in FIG. 4, the inclined flat or face 2 permits as a first step, to provide some play which makes it possible to insert the screwdriver 1 into the pentagonal recess or at five socket faces E of the head of the screw V.

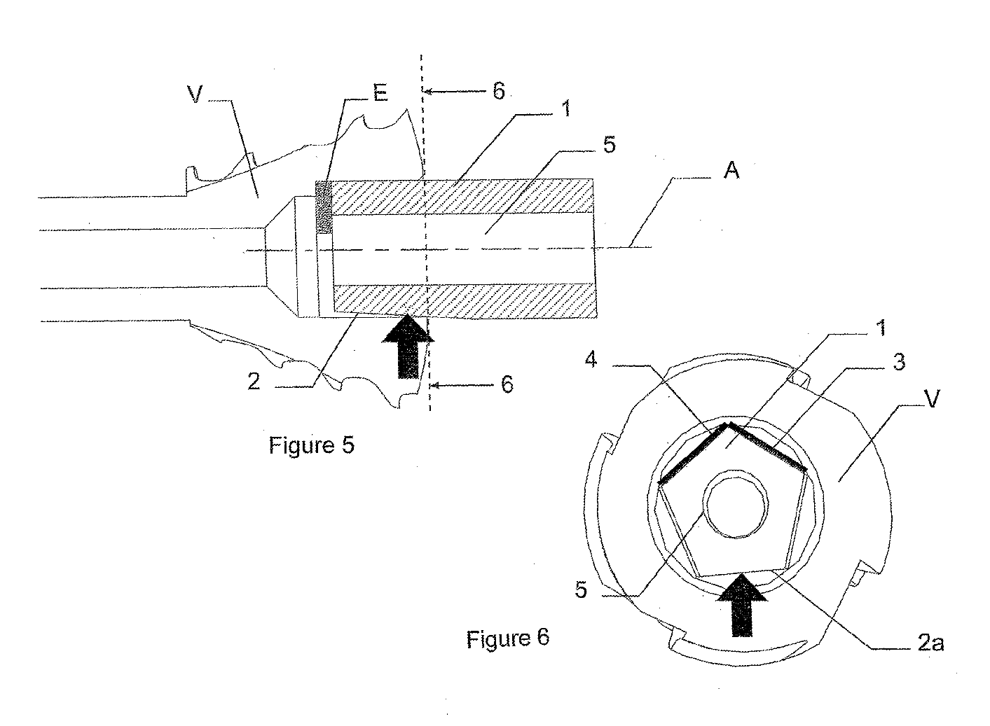

As the screwdriver 1 is inserted into the recess E of the screw head, the play diminishes and disappears altogether, thus permitting the screwdriver to perfectly match up with the shape of the recess E of the screw V (FIG. 5).

Furthermore, when the screwdriver is completely inserted into the recess E of the head of screw V, the inclined face 2 makes it possible to place the two faces or flats 3 and 4, opposite the inclined flat 2, closely against the corresponding socket faces of the recess of the screw, as indicated by the arrow on FIGS. 5 and 6.

The screwdriver 1 then finds itself immobilized in the head of the screw V and the inclined flat creates a gripping effect.

Thus the stress applied to the screwdriver 1 to screw or unscrew an orthopedic screw is transferred to said screw through the intermediary of the edge 2a of the inclined flat 2 by the faces 3 and 4 opposite to said inclined flat 2 and by the angle comprised between said faces 3 and 4.

The pentagon of the screwdriver 1 being convex and regular, its interior angles amount to 108° which permits a better transfer of the stress than that obtained by the means of screw work systems using socket hex screws and screwdrivers with angles of 120°.

Utilization of this geometric shape is also remarkable in that the surface of the circle in which the pentagon is inscribed and which is not occupied by the pentagon is larger than for a hexagon.

In this way, any manifestation of the shifting phenomenon leading to a deformation of the material of the head of the orthopedic screw V is eliminated.

The invention also concerns a screw work system including screws with pentagonal recess and a screwdriver featuring one or several of the characteristic arrangements previously described and disclosed in the claims.

1. Pentagonal screwdriver, for screwing of screws with pentagonal recess, in particular of orthopedic screws, characterized in that it has one inclined flat relative to and in direction of the longitudinal axis of said screwdriver, the other four flats of the screwdriver being straight and parallel to said longitudinal axis.

2. Pentagonal screwdriver according to claim 1, characterized in that the inclination of the flat is between 2° and 3°.

3. Pentagonal screwdriver according to claim 1, characterized in that the flat is inclined by 2° for being used with screws featuring a head of a diameter of 2 mm or less than 2 mm.

4. Pentagonal screwdriver according to claim 1, characterized in that the flat is inclined by 3° for being used with screws featuring a head of a diameter of more than 2 mm.

5. Pentagonal screwdriver according to claim 1, characterized in that it is provided with an axial bore.

6. Pentagonal screwdriver according to claim 1, characterized in that it is made of any so-called surgical material presenting the necessary hardness.

7. Pentagonal screwdriver according to claim 1, characterized in that is constructed of stainless steel.

8. Pentagonal screwdriver according to claim 1, characterized in that it is constructed of stainless steel having undergone a heat treatment for the purpose of increasing its hardness.

9. System of screw work comprising a screwdriver according to claim 1 and screws with pentagonal recess.
